(19) United States
(12) Patent Application Publication
(10) Pub. No.: US 2025/0054589 A1
(43) Pub. Date: Feb. 13, 2025

(54) DIAGNOSTIC ARTIFICIAL INTELLIGENCE ASSISTANT

(71) Applicant: Medical Security Card Company, LLC, Tucson, AZ (US)

(72) Inventors: Jon Olaf JENSEN, New Canaan, CT (US); Marcus Nicholas SREDZINSKI, Tucson, AZ (US)

(21) Appl. No.: 18/799,372

(22) Filed: Aug. 9, 2024

Related U.S. Application Data
(60) Provisional application No. 63/532,214, filed on Aug. 11, 2023.

(51) Int. Cl. G16H 10/60

(52) U.S. Cl. CPC G16H 10/60

(57) ABSTRACT

A user system receives a request for medical information about a first patient, parses the request to identify the first patient, analyzes the request to identify one or more dataset categories corresponding to the request, and generates one or more queries based on the request, each query corresponding to an identified dataset category. The user system sends each of the one or more queries to at least one of a plurality of data repositories comprising patient medical information related to the identified dataset category corresponding to the respective query. The user system receives a response to each of the one or more queries and generates a response to the request for medical information about the first patient, the response identifying one of the plurality of data repositories as the source of each portion of medical information about the first patient.

### RELATED APPLICATION

The present application claims priority to U.S. patent application No. 63/532,214 filed 11 Aug. 2023, which is incorporated herein by reference in its entirety.

### BACKGROUND

### Field of the Invention

The present disclosure is generally related to patient medical information and more specifically related to providing patient medical information from disparate authoritative sources to medical personnel.

### Related Art

Conventional systems store patient medical information in a variety of separate archives that generally have their own unique formats and are related to particular categories of medical services. This type of distributed storage of patient medical information frustrates the ability for medical professionals to provide holistic treatment. What is needed is a system and method that overcomes these significant problems found in the conventional systems for storing patient medical information as described above.

### SUMMARY

Accordingly, described herein are solutions to the problems in the conventional commercial transactions described above. In some aspects, the techniques described herein relate to a system for providing patient medical information, the system including: a server system communicatively coupled with a plurality of data repositories via a data communication network, each data repository including patient medical information related to at least one dataset category, the server system configured to: receive a request for medical information about a first patient; parse the request to identify the first patient; analyze the request to identify one or more dataset categories corresponding to the request; generate one or more queries based on the request, each query corresponding to an identified dataset category; send each of the one or more queries to at least one of the plurality of data repositories including patient medical information related to the identified dataset category corresponding to the respective query; receive a response to each of the one or more queries, each response including patient medical information about the first patient related to the identified dataset category corresponding to the respective query; generate a response to the request for medical information about the first patient, the response including one or more of the received responses to each of the one or more queries; and for each of the one or more of the received responses to each of the one or more queries included in the response to the request for medical information about the first patient, identify one of the plurality of data repositories as a data repository source of the patient medical information about the first patient included in the response to the request for medical information about the first patient.

In some aspects, the techniques described herein relate to a system, wherein the server system is further configured to receive the request from a user system via the data communication network.

In some aspects, the techniques described herein relate to a system, wherein the server system is further configured to provide the response to the user system via the data communication network.

In some aspects, the techniques described herein relate to a system, wherein the server system is further configured to generate a summary report of the one or more of the received responses to each of the one or more queries.

In some aspects, the techniques described herein relate to a system, wherein the server system is further configured to reformat one or more of the one or more received responses to each of the one or more queries into a common format.

In some aspects, the techniques described herein relate to a system, wherein the common format includes the patient medical information about the first patient related to the identified dataset category corresponding to the respective query and the data repository source corresponding to said patient medical information.

In some aspects, the techniques described herein relate to an apparatus for providing patient medical information, including: a non-transitory computer readable medium configured to store executable programmed modules; a processor communicatively coupled with the non-transitory computer readable medium and configured to execute programmed modules stored in the non-transitory computer readable medium, the processor further configured to, receive a request for medical information about a first patient; parse the request to identify the first patient; analyze the request to identify one or more dataset categories corresponding to the request; generate one or more queries based on the request, each query corresponding to an identified dataset category; send each of the one or more queries to at least one data repository including medical information related to the identified dataset category corresponding to the respective query; receive a response to each of the one or more queries, each response including patient medical information about the first patient related to the identified dataset category corresponding to the respective query; generate a response to the request for medical information about the first patient, the response including one or more of the received responses to each of the one or more queries; and for each of the received responses to each of the one or more queries included in the response to the request for medical information about the first patient, identify one of the at least one data repository as a data repository source of the patient medical information about the first patient included in the response to the request for medical information about the first patient.

In some aspects, the techniques described herein relate to an apparatus, wherein the processor is further configured to receive the request from a user system via a data communication network.

In some aspects, the techniques described herein relate to an apparatus, wherein the processor is further configured to provide the response to the user system via the data communication network.

In some aspects, the techniques described herein relate to an apparatus, wherein the processor is further configured to generate a summary report of the one or more of the received responses to each of the one or more queries.

In some aspects, the techniques described herein relate to an apparatus, wherein the processor is further configured to reformat one or more of the one or more received responses to each of the one or more queries into a common format.

In some aspects, the techniques described herein relate to an apparatus, wherein the common format includes the patient medical information about the first patient related to the identified dataset category corresponding to the respective query and the data repository source corresponding to said patient medical information.

In some aspects, the techniques described herein relate to a computer implemented method for providing patient medical information, where one or more processors are programmed to perform steps including: receive a request for medical information about a first patient; parse the request to identify the first patient; analyze the request to identify one or more dataset categories corresponding to the request; generate one or more queries based on the request, each query corresponding to an identified dataset category; send each of the one or more queries to at least one data repository including medical information related to the identified dataset category corresponding to the respective query; receive a response to each of the one or more queries, each response including patient medical information about the first patient related to the identified dataset category corresponding to the respective query; generate a response to the request for medical information about the first patient, the response including one or more of the received responses to each of the one or more queries; and for each of the received responses to each of the one or more queries included in the response to the request for medical information about the first patient, identify one of the at least one data repository as a data repository source of the patient medical information about the first patient included in the response to the request for medical information about the first patient.

In some aspects, the techniques described herein relate to a method, wherein the one or more processors are further programmed to receive the request from a user system via a data communication network.

In some aspects, the techniques described herein relate to a method, wherein the one or more processors are further programmed to provide the response to the user system via the data communication network.

In some aspects, the techniques described herein relate to a method, wherein the one or more processors are further programmed to generate a summary report of the one or more of the received responses to each of the one or more queries.

In some aspects, the techniques described herein relate to a method, wherein the one or more processors are further programmed to reformat one or more of the one or more received responses to each of the one or more queries into a common format.

In some aspects, the techniques described herein relate to a method, wherein the common format includes the patient medical information about the first patient related to the identified dataset category corresponding to the respective query and the data repository source corresponding to said patient medical information.

In some aspects, the techniques described herein relate to a non-transitory computer readable medium having stored thereon one or more sequences of instructions for causing one or more processors to perform steps for providing patient medical information, including: receive a request for medical information about a first patient; parse the request to identify the first patient; analyze the request to identify one or more dataset categories corresponding to the request; generate one or more queries based on the request, each query corresponding to an identified dataset category; send each of the one or more queries to at least one data repository including medical information related to the identified dataset category corresponding to the respective query; receive a response to each of the one or more queries, each response including patient medical information about the first patient related to the identified dataset category corresponding to the respective query; generate a response to the request for medical information about the first patient, the response including one or more of the received responses to each of the one or more queries; and for each of the received responses to each of the one or more queries included in the response to the request for medical information about the first patient, identify one of the at least one data repository as a data repository source of the patient medical information about the first patient included in the response to the request for medical information about the first patient.

In some aspects, the techniques described herein relate to a system including at least one processor communicatively coupled with at least one non-transitory computer readable medium, wherein the at least one processor is programmed to: receive a request for medical information about a first patient; parse the request to identify the first patient; analyze the request to identify one or more dataset categories corresponding to the request; generate one or more queries based on the request, each query corresponding to an identified dataset category; send each of the one or more queries to at least one data repository including medical information related to the identified dataset category corresponding to the respective query; receive a response to each of the one or more queries, each response including patient medical information about the first patient related to the identified dataset category corresponding to the respective query; generate a response to the request for medical information about the first patient, the response including one or more of the received responses to each of the one or more queries; and for each of the received responses to each of the one or more queries included in the response to the request for medical information about the first patient, identify one of the at least one data repository as a data repository source of the patient medical information about the first patient included in the response to the request for medical information about the first patient.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment; and

FIG. 3 illustrates an example process for providing patient medical information, according to an embodiment.

### DETAILED DESCRIPTION

Disclosed herein are systems, methods, and non-transitory computer-readable media for providing patient medical information. For example, one method disclosed herein allows for a system to receive a first query corresponding to a particular patient and analyze the request to identify one or more categories of requested patient medical information. Based on the patient identification and the requested categories of medical information, the system generates one or more second queries, each second query customized for a particular repository of patient medical information. The system sends each second query to the corresponding repository of patient medical information and receives a response to each of the second queries. The system compiles the responses into a response to the first query and identifies the particular repository of patient medical information that provided each portion of data in the response to the first query.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### 1. System Overview

### 1.1. Example Infrastructure

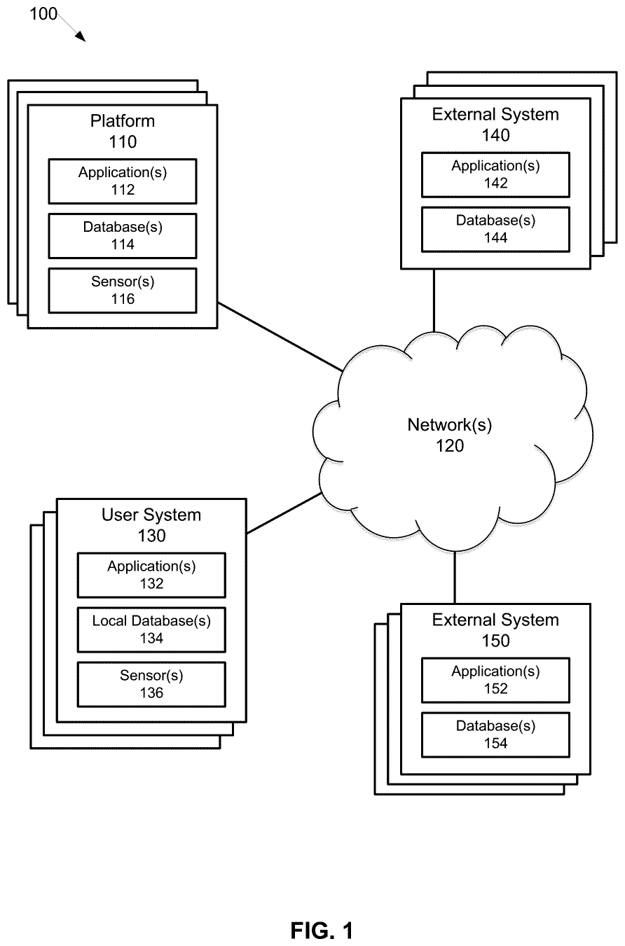

FIG. 1 illustrates an example infrastructure in which one or more of the disclosed processes may be implemented, according to an embodiment. The infrastructure may comprise a platform 110 (e.g., one or more server computers or point of sale (POS) devices) which hosts and/or executes one or more of the various functions, processes, methods, and/or software modules described herein. Platform 110 may comprise dedicated hardware and software, or may instead comprise cloud instances, which utilize shared resources of one or more servers. These POS devices, servers, and/or cloud instances may be collocated and/or geographically distributed. Platform 110 may also comprise or be communicatively connected to an application 112 and/or one or more databases 114. In addition, platform 110 may be communicatively connected to one or more user systems 130 via one or more networks 120. Platform 110 may also be communicatively connected to one or more external systems 140 and 150 (e.g., other platforms, websites, information repositories, databases, software-as-a-service platforms, etc.) via one or more networks 120.

Network(s) 120 may comprise a personal area network, a mesh network, a local area network, a wide area network, the Internet, and any combination of wired or wireless networks. Networks(s) 120 may also include direct wired or wireless connections such as Bluetooth®. Platform 110 may communicate with user system(s) 130 through the network 120 using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via a private network such as an intranet or a direct communication link such as Bluetooth®. Furthermore, while only a few of each of the user systems 130, external systems 140, external systems 150, and platforms 110, are illustrated, it should be understood that the infrastructure may comprise any number of platforms, user systems, and external systems and any number of applications, databases, and sensors.

User system(s) 130 may comprise any type or types of computing devices capable of wired and/or wireless communication, including without limitation, desktop computers, laptop computers, tablet computers, smart phones or other mobile phones, servers, game consoles, head mounted displays, televisions, set-top boxes, electronic kiosks, point-of-sale terminals, Automated Teller Machines, and/or the like. In one aspect, user system 130 is a wireless communication device such as a smart phone that includes a sensor 136 such as a camera and the ability to communicate via a WIFI connection and also a cellular network connection.

External systems 140 and 150 may comprise web servers which host one or more websites and/or web services. For example, external systems 140 and 150 may comprise one or more databases 144 and 154 that comprise medical information about a patient such as medical history, prior and current prescriptions, and the like. Additionally, external systems 140 and 150 may be affiliated with a hospital or doctor's office or may be affiliated with a third party service such as a telemedicine service provider.

In embodiments in which a website is provided by one or more of external systems 140 and 150, the website may comprise a graphical user interface, including, for example, one or more screens (e.g., webpages) generated in HyperText Markup Language (HTML) or other language. External systems 140 and 150 may also provide software-as-a-service capabilities and respond to queries that are received from, for example, platform 110 and/or user system 130. In doing so, external systems 140 and 150 may transmit or serve one or more screens of the graphical user interface in response to requests from platform 110 and/or user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to external systems 140 and 150 and the responses from external systems 140 and 150, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 144 and 154) that are locally and/or remotely accessible to external systems 140 and 150. External systems 140 and 150 may also respond to other requests from platform 110 and/or user system(s) 130.

Platform 110 may further comprise, be communicatively coupled with, or otherwise have access to one or more database(s) 114. For example, platform 110 may comprise one or more database servers which manage one or more databases 114. A user system 130 or application 112 executing on platform 110 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, external systems 140 and 150 may receive requests from platform 110 and/or user system 130, and provide responses in extensible Markup Language (XML), JavaScript Object Notation (JSON), and/or any other suitable or desired format. In such embodiments, external systems 140 and 150 may provide an application programming interface (API) which defines the manner in which platform 110 and/or user system(s) 130 may interact with the web service. Thus, platform 110 and/or user system(s) 130, can define their own user interfaces, and rely on the web service to implement or otherwise provide the backend processes, methods, functionality, storage, and/or the like, described herein. For example, in such an embodiment, a client application 132 executing on one or more user system(s) 130 may interact with an application 142 or application 152 executing on external systems 140 and 150 to execute one or more or a portion of one or more of the various functions, processes, methods, and/or software modules described herein.

On the platform 110 and user system 130, application 112 and application 132 may be “thin,” in which case processing is primarily carried out server-side by applications 142 or 152 on external systems 140 and 150. A basic example of a thin client application 112 and/or 132 is a browser application, which simply requests, receives, and renders webpages at platform 110 and/or user system(s) 130, while server applications 142 and/or 152 on external systems 140 and 150 are responsible for generating the webpages and managing database functions. Alternatively, the client application may be “thick,” in which case processing is primarily carried out client-side by platform 110 or user system(s) 130. It should be understood that application 112 and/or application 132 may perform an amount of processing, relative to a server application 142 and/or 152 on external systems 140 and 150, at any point along this spectrum between “thin” and “thick,” depending on the design goals of the particular implementation. In any case, the application described herein, which may wholly reside on user system(s) 130 or be distributed between platform 110 and user system(s) 130 (e.g., in which case application 112 and application 132 both perform processing), can comprise one or more executable software modules that implement one or more of the processes, methods, or functions of the application described herein.

### 1.2. Example Processing Device

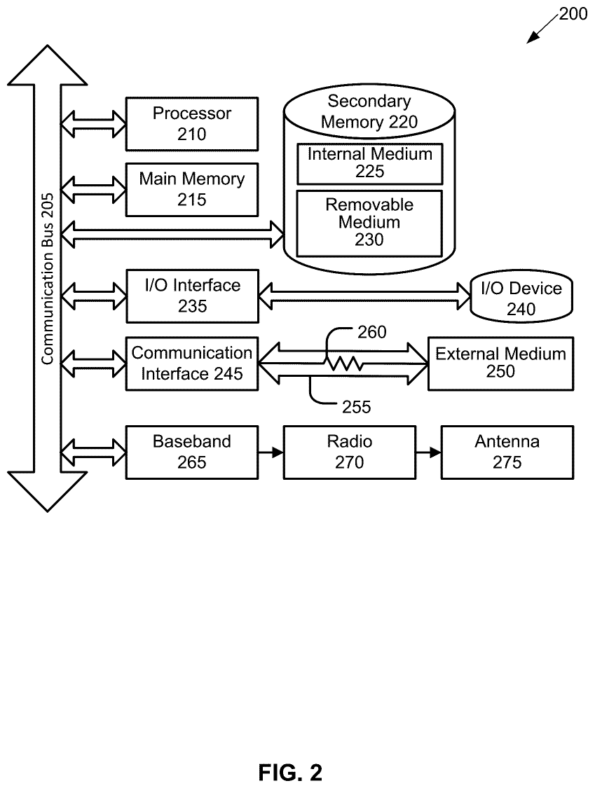

FIG. 2 is a block diagram illustrating an example wired or wireless system 200 that may be used in connection with various embodiments described herein. For example, system 200 may be used as or in conjunction with one or more of the functions, processes, or methods (e.g., to store and/or execute the application or one or more software modules of the application) described herein, and may represent components of platform 110, user system(s) 130, external system(s) 140, and/or other processing devices described herein. System 200 can be a smartphone device, a tablet computer, or any other processor-enabled device that is capable of wired or wireless data communication and preferable capable of data communication on a WIFI network and also on a cellular network. Other computer systems and/or architectures may be also used, as will be clear to those skilled in the art.

System 200 preferably includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, the Pentium® processor, Core i7® processor, and Xeon® processor, all of which are available from Intel Corporation of Santa Clara, California.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 preferably includes a main memory 215 and may also include a secondary memory 220. Main memory 215 provides storage of instructions and data for programs executing on processor 210, such as one or more of the functions and/or modules discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 220 is a non-transitory computer-readable medium having computer-executable code (e.g., disclosed software modules) and/or other data stored thereon. The computer software or data stored on secondary memory 220 is read into main memory 215 for execution by processor 210.

In alternative embodiments, secondary memory 220 may include other similar means for allowing computer programs or other data or instructions to be loaded into system 200. Such means may include, for example, a communication interface 245, which allows software and data to be transferred from external storage medium 250 to system 200. Examples of external storage medium 250 may include an external hard disk drive, an external optical drive, an external magneto-optical drive, and/or the like. Other examples of secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 200 may include a communication interface 245. Communication interface 245 allows software and data to be transferred between system 200 and external devices (e.g. printers), networks, or other information sources. For example, computer software or executable code may be transferred to system 200 from a network server (e.g., platform 110) via communication interface 245. Examples of communication interface 245 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 245 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 245 are generally in the form of electrical communication signals 260. These signals 260 may be provided to communication interface 245 via a communication channel 255. In an embodiment, communication channel 255 may be a wired or wireless network (e.g., network(s) 120), or any variety of other communication links. Communication channel 255 carries signals 260 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 215 and/or secondary memory 220. Computer programs can also be received via communication interface 245 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 250), and any peripheral device communicatively coupled with communication interface 245 (including a network information server or other network device). These non-transitory computer-readable media are means for providing executable code, programming instructions, software, and/or other data to system 200.

In an embodiment that is implemented using software, the software may be stored on a computer-readable medium and loaded into system 200 by way of removable medium 230, I/O interface 235, or communication interface 245. In such an embodiment, the software is loaded into system 200 in the form of electrical communication signals 260. The software, when executed by processor 210, preferably causes processor 210 to perform one or more of the processes and functions described elsewhere herein.

In an embodiment, I/O interface 235 provides an interface between one or more components of system 200 and one or more input and/or output devices 240. Example input devices include, without limitation, sensors, cameras, microphones, keyboards, touch screens or other touch-sensitive devices, biometric sensing devices, computer mice, trackballs, pen-based pointing devices, and/or the like. Examples of output devices include, without limitation, other processing devices, speakers, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), head mounted displays (HMDs), and/or the like. In some cases, an input and output device 240 may be combined, such as in the case of a touch panel display (e.g., in a smartphone, tablet, or other mobile device).

In an embodiment, the I/O device 240 may be any type of external or integrated display and may include one or more discrete displays that in aggregate form the I/O device 240. The I/O device 240 may be capable of 2D or 3D presentation of visual information to a user of the system 200. In one embodiment, the I/O device 240 may be a virtual reality or augmented reality device in the form of HMD by the user so the user may visualize the presentation of information in 3D.

System 200 may also include optional wireless communication components that facilitate wireless communication over a voice network and/or a data network (e.g., in the case of user system 130). The wireless communication components comprise an antenna system 275, a radio system 270, and a baseband system 265. In system 200, radio frequency (RF) signals are transmitted and received over the air by antenna system 275 under the management of radio system 270.

In an embodiment, antenna system 275 may comprise one or more antennae and one or more multiplexors (not shown) that perform a switching function to provide antenna system 275 with transmit and receive signal paths. In the receive path, received RF signals can be coupled from a multiplexor to a low noise amplifier (not shown) that amplifies the received RF signal and sends the amplified signal to radio system 270.

In an alternative embodiment, radio system 270 may comprise one or more radios that are configured to communicate over various frequencies. In an embodiment, radio system 270 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 270 to baseband system 265.

If the received signal contains audio information, then baseband system 265 decodes the signal and converts it to an analog signal. Then the signal is amplified and sent to a speaker. Baseband system 265 also receives analog audio signals from a microphone. These analog audio signals are converted to digital signals and encoded by baseband system 265. Baseband system 265 also encodes the digital signals for transmission and generates a baseband transmit audio signal that is routed to the modulator portion of radio system 270. The modulator mixes the baseband transmit audio signal with an RF carrier signal, generating an RF transmit signal that is routed to antenna system 275 and may pass through a power amplifier (not shown). The power amplifier amplifies the RF transmit signal and routes it to antenna system 275, where the signal is switched to the antenna port for transmission.

Baseband system 265 is also communicatively coupled with processor 210, which may be a central processing unit (CPU). Processor 210 has access to data storage areas 215 and 220. Processor 210 is preferably configured to execute instructions (i.e., computer programs, such as the disclosed application, or software modules) that can be stored in main memory 215 or secondary memory 220. Computer programs can also be received from baseband processor 260 and stored in main memory 210 or in secondary memory 220, or executed upon receipt. Such computer programs, when executed, enable system 200 to perform the various functions of the disclosed embodiments.

### 1.3. Example Operation

Referring back to FIG. 1, in one aspect, the system 100 operates such that the user system 130 is operated by a medical professional and the user system 130 collects information about the patient and one or more categories of medical information that is desired. The user system 130 sends a request for the desired medical information to the platform 110. The platform 110 parses the request to identify the patient and one or more categories of medical information that is desired. The platform 1110 constructs one or more queries corresponding to the patient and the one or more categories. For example, the platform 110 may generate one query for each category of medical information. Alternatively, the platform 110 may generate one query for each of a plurality of data repositories, where each query requests a particular category of medical information. Each query identifies the patient and the type of medical information that is desired. Once the queries are generated, the platform 110 sends each of the queries to at least one external system 140 or 150. The platform 110 receives responses from the various external systems 140 and 150 and generates a response to the original request received from the user system 130. When generating the response, the platform 110 may reformat the data received from the one or more external systems 140 and 150. Additionally, when generating the response, the platform 110 identifies the particular external system 140 as the source of each element of patient medical information that is included in the report.

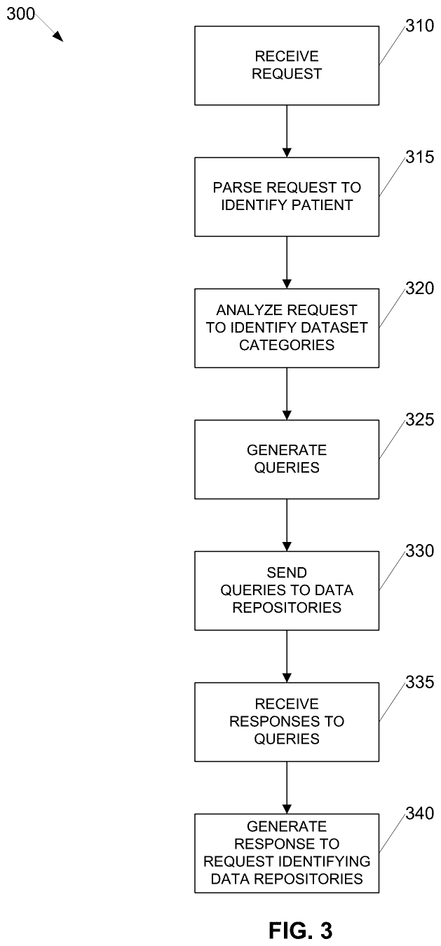

FIG. 3 illustrates an example process 300 for providing patient medical information, according to an embodiment. In one aspect, the process 300 may be carried out by the system described with respect to FIG. 1 in combination with one of more processing devices described with respect to FIG. 2.

Initially, at 310 the platform 110 receives a request for patient medical information about a particular patient. The request may be received via a network interface, for example via an API or a web service hosted by the platform 110. The request may also be received from an application 132 executing on a user system 130 in cooperation with an application 112 executing on the platform.

At 315 the platform 110 parses the request to identify the patient for which the medical information has been requested. Additionally, at 320, the platform 110 analyzes the request to identify one or more categories of medical information that is desired and/or one or more sub-categories of medical information that is desired. As will be understood by the skilled artisan, multiple levels of category and sub-category may be present in a single request. In one aspect, a category of medical information may correspond to a particular medical field and/or a collection of medical information. For example, the request may be seeking all prior surgeries for the patient, or all allergies of the patient, or all current prescriptions of the patient, or all prior radiology scans of the patient, just to name a few.

Next, at 325 the platform 110 generates one or more queries. Each query identifies the patient and also identifies the category (or sub-category or sub-sub-category, etc.) of medical information that is desired. In one aspect, a first external system 140 may store patient medical information for, e.g., specific anesthesia drugs and quantities administered to the patient during general anesthesia surgery, while a second external system 150 may store patient medical information for, e.g., general anesthesia surgical procedures performed on the patient without reference to the specific anesthesia drugs and quantities. Advantageously, the queries can be generated in a format corresponding to a particular external system 140 or 150. For example, each individual query may be formatted according to a defined application programming interface (API) or may be formatted according to a defined uniform resource locator (URL). Advantageously, the platform 110 may construct the one or more queries in parallel so that they may be simultaneously sent to each of a plurality of external systems 140 or 150.

Next, at 330 the one or more queries are sent by the platform to one or more external systems 140 or 150, where the queries may be processed in parallel by each respective external system 140 or 150. Such parallel processing of queries significantly reduces the overall transaction time for obtaining authoritative patient medical information that can be reviewed by medical care professionals. In one aspect, the platform 110 sends each query to an external system 140 or 150 via a data communication network 120, which may include the Internet.

Next, at 335 the platform 110 receives a response to each of the queries sent to each of the many external systems 140 or 150. The platform 110 analyzes each response and reformats the data elements provided in each response so that all data elements received across all responses have a common format. The platform 110 also constructs a summary report of the responses to each of the queries and in the summary report, the platform 110 identifies the external system 140 or 150 that provided each data element included in the summary report.

Next, at 340, the platform 110 generates a response to the original request received at 310. For example, the platform 110 may include the summary report in the response to the original request. In one aspect, the platform 110 presents the summary report in a user interface of the platform 110. Alternatively, the platform 110 may send the summary report to a user system 130 for presentation on a user interface of the user system 130.

Advantageously, the summary report provides to the requesting medical professionals both the requested patient medical information and also a reference to the authoritative source of the patient medical information. Providing both the requested patient medical information and the reference to the authoritative source of the patient medical information allows the requesting medical professionals to evaluate the veracity of the patient medical information based on the source of the information.

In one aspect, constructing the queries in parallel and sending the queries to the many external systems 140 or 150 for processing in parallel and reformatting the various responses into a common format and providing a summary of the responses that designates the source of each element in the summary of the responses significantly reduces the time for medical professionals to obtain and evaluate patient medical information and improves the quality and speed of providing medical care. By significantly reducing the time required to consult with and treat patients, the system 100 improves the overall operation of medical service providers who require authoritative patient medical information in advance of providing medical care.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A system for providing patient medical information, the system comprising:
a server system communicatively coupled with a plurality of data repositories via a data communication network, each data repository comprising patient medical information related to at least one dataset category, the server system configured to:
receive a request for medical information about a first patient;
parse the request to identify the first patient;
analyze the request to identify one or more dataset categories corresponding to the request;
generate one or more queries based on the request, each query corresponding to an identified dataset category;
send each of the one or more queries to at least one of the plurality of data repositories comprising patient medical information related to the identified dataset category corresponding to the respective query;
receive a response to each of the one or more queries, each response including patient medical information about the first patient related to the identified dataset category corresponding to the respective query;
generate a response to the request for medical information about the first patient, the response comprising one or more of the received responses to each of the one or more queries; and
for each of the one or more of the received responses to each of the one or more queries included in the response to the request for medical information about the first patient, identify one of the plurality of data repositories as a data repository source of the patient medical information about the first patient included in the response to the request for medical information about the first patient.

2. The system of claim 1, wherein the server system is further configured to receive the request from a user system via the data communication network.

3. The system of claim 2, wherein the server system is further configured to provide the response to the user system via the data communication network.

4. The system of claim 1, wherein the server system is further configured to generate a summary report of the one or more of the received responses to each of the one or more queries.

5. The system of claim 1, wherein the server system is further configured to reformat one or more of the one or more received responses to each of the one or more queries into a common format.

6. The system of claim 5, wherein the common format comprises the patient medical information about the first patient related to the identified dataset category corresponding to the respective query and the data repository source corresponding to said patient medical information.

7. An apparatus for providing patient medical information, comprising:
a non-transitory computer readable medium configured to store executable programmed modules;
a processor communicatively coupled with the non-transitory computer readable medium and configured to execute programmed modules stored in the non-transitory computer readable medium, the processor further configured to, receive a request for medical information about a first patient;
parse the request to identify the first patient;
analyze the request to identify one or more dataset categories corresponding to the request;
generate one or more queries based on the request, each query corresponding to an identified dataset category;
send each of the one or more queries to at least one data repository comprising medical information related to the identified dataset category corresponding to the respective query;
receive a response to each of the one or more queries, each response including patient medical information about the first patient related to the identified dataset category corresponding to the respective query;
generate a response to the request for medical information about the first patient, the response comprising one or more of the received responses to each of the one or more queries; and
for each of the received responses to each of the one or more queries included in the response to the request for medical information about the first patient, identify one of the at least one data repository as a data repository source of the patient medical information about the first patient included in the response to the request for medical information about the first patient.

8. The apparatus of claim 7, wherein the processor is further configured to receive the request from a user system via a data communication network.

9. The apparatus of claim 8, wherein the processor is further configured to provide the response to the user system via the data communication network.

10. The apparatus of claim 7, wherein the processor is further configured to generate a summary report of the one or more of the received responses to each of the one or more queries.

11. The apparatus of claim 7, wherein the processor is further configured to reformat one or more of the one or more received responses to each of the one or more queries into a common format.

12. The apparatus of claim 11, wherein the common format comprises the patient medical information about the first patient related to the identified dataset category corresponding to the respective query and the data repository source corresponding to said patient medical information.

13. A computer implemented method for providing patient medical information, where one or more processors are programmed to perform steps comprising:
receive a request for medical information about a first patient;
parse the request to identify the first patient;
analyze the request to identify one or more dataset categories corresponding to the request;
generate one or more queries based on the request, each query corresponding to an identified dataset category;
send each of the one or more queries to at least one data repository comprising medical information related to the identified dataset category corresponding to the respective query;
receive a response to each of the one or more queries, each response including patient medical information about the first patient related to the identified dataset category corresponding to the respective query;
generate a response to the request for medical information about the first patient, the response comprising one or more of the received responses to each of the one or more queries; and
for each of the received responses to each of the one or more queries included in the response to the request for medical information about the first patient, identify one of the at least one data repository as a data repository source of the patient medical information about the first patient included in the response to the request for medical information about the first patient.

14. The method of claim 13, wherein the one or more processors are further programmed to receive the request from a user system via a data communication network.

15. The method of claim 14, wherein the one or more processors are further programmed to provide the response to the user system via the data communication network.

16. The method of claim 13, wherein the one or more processors are further programmed to generate a summary report of the one or more of the received responses to each of the one or more queries.

17. The method of claim 13, wherein the one or more processors are further programmed to reformat one or more of the one or more received responses to each of the one or more queries into a common format.

18. The method of claim 17, wherein the common format comprises the patient medical information about the first patient related to the identified dataset category corresponding to the respective query and the data repository source corresponding to said patient medical information.

19. A non-transitory computer readable medium having stored thereon one or more sequences of instructions for causing one or more processors to perform steps for providing patient medical information, comprising:
receive a request for medical information about a first patient;
parse the request to identify the first patient;
analyze the request to identify one or more dataset categories corresponding to the request;
generate one or more queries based on the request, each query corresponding to an identified dataset category;
send each of the one or more queries to at least one data repository comprising medical information related to the identified dataset category corresponding to the respective query;
receive a response to each of the one or more queries, each response including patient medical information about the first patient related to the identified dataset category corresponding to the respective query;
generate a response to the request for medical information about the first patient, the response comprising one or more of the received responses to each of the one or more queries; and
for each of the received responses to each of the one or more queries included in the response to the request for medical information about the first patient, identify one of the at least one data repository as a data repository source of the patient medical information about the first patient included in the response to the request for medical information about the first patient.

20. A system comprising at least one processor communicatively coupled with at least one non-transitory computer readable medium, wherein the at least one processor is programmed to:
receive a request for medical information about a first patient;
parse the request to identify the first patient;
analyze the request to identify one or more dataset categories corresponding to the request;
generate one or more queries based on the request, each query corresponding to an identified dataset category;
send each of the one or more queries to at least one data repository comprising medical information related to the identified dataset category corresponding to the respective query;
receive a response to each of the one or more queries, each response including patient medical information about the first patient related to the identified dataset category corresponding to the respective query;
generate a response to the request for medical information about the first patient, the response comprising one or more of the received responses to each of the one or more queries; and
for each of the received responses to each of the one or more queries included in the response to the request for medical information about the first patient, identify one of the at least one data repository as a data repository source of the patient medical information about the first patient included in the response to the request for medical information about the first patient.
